(19) United States
(12) Patent Application Publication
(10) Pub. No.: US 2015/0320623 A1
(43) Pub. Date: Nov. 12, 2015

(54) POST-SURGICAL DRAIN MANAGEMENT GARMENT

(71) Applicant: Life in the Pink, Inc., Atlanta, GA (US)

(72) Inventors: Tonya E. Lee, Atlanta, GA (US); Ismary Susset Bryan, Suwanee, GA (US)

(73) Assignee: Life in the Pink, Inc., Atlanta, GA (US)

(21) Appl. No.: 14/273,296

(22) Filed: May 8, 2014

(51) Int. Cl. A61F 15/00

(52) U.S. Cl. CPC A61F 15/008

(57) ABSTRACT

A post-surgical drain management garment comprises a lightweight, disposable, wrap-around material with pouches for comfortable placement of surgical drains. The garment provides a wrap-around piece designed to keep the post-surgical drains secure while in the hospital, post-surgical facility, or other recovery facility. The garment is wrapped around the torso, arm, leg, or other body part of the patient and then closed with a secure closure. A post-surgical drain is slipped over the top or bottom of the garment and placed in a pouch.

### TECHNICAL FIELD

The present invention relates generally to post-surgical garments and more specifically to a wrap-around, drain management garment for post-surgical drains for patients who have had surgery.

### BACKGROUND

After surgeries, patients often have implanted surgical drains. The drains remove content of body organs and secretions of the body cavity, such as blood, serum, lymph, and other body fluids that may accumulate in or around a wound after a surgical procedure. Depending on the amount of drainage, a patient may have the drain in place one day to multiple weeks.

Pursuant to the drain manufacturer's suggestion, patients and hospital staff are instructed to pin drains to the patient's clothing. Alternatively, the drains are attached to, or simply laid in, the hospital bed with the patient. When a patient needs to move (for example, to use the restroom or attend physical therapy), the drains are then attached to an intravenous therapy (IV) pole or held by the hospital staff.

### SUMMARY

In certain example aspects described herein, a post-surgical garment comprises a wrap-around design that comprises pouches for comfortable placement of post-surgical drains. In an example embodiment, the garment specifically meets the needs of patients who have any surgeries that require post-surgical drains. The garment provides one solid wrap around piece designed to keep the post-surgical drains secure while the patient is recovering in the hospital or therapeutic/recovery location. In an example embodiment, the garment allows for security of the post-surgical drains while the patient is laying, sitting, or lounging in a bed or chair, standing, and/or walking. In another example embodiment, the garment securely holds the post-surgical drain bulbs to protect against movement and possible dislodging of the drains. In an example embodiment, the garment comprises a disposable or limited use material that is used or worn for a limited amount of time. In another example embodiment, the garment is changed on a regular basis, for example, hourly or daily. In an example embodiment, the garment comprises a top and bottom closure that secures the garment to the body of the patient. In another example embodiment, the garment is a loose-fitting, solid, wrap-around piece designed to hold the post-surgical drains. In another example embodiment, the garment is designed to hold post-surgical drains, catheters, colostomy bags, collection bags, collection containers, and/or tubing.

In an example embodiment, the garment is worn around the torso or waist of the patient. In another example embodiment, the garment is worn around features of the body, such as the patient's arms, legs, thighs, calves, knees, or other joints in the body.

In an example embodiment, the garment is used by wrapping the garment around the patient's body. The garment is then closed in the front, side, back, top, or bottom with a secure closure. A post-surgical drain is slipped over the top or bottom edge of the garment. The post-surgical drain and excess tubing is placed in a pouch of the garment.

In an example embodiment, the garment is a recovery or lounging garment made of a light weight, disposable fabric/material, such as SMS, Tyvek, poly-Lycra fabric blend, cotton, or other breathable material that is worn for daily use to secure post-surgical drains. In an example embodiment, the garment does not provide compression of a wound area. In another example embodiment, the garment does not cover or protect the wound area. In an example embodiment, the garment is made of a material that is lightweight and disposable, but sturdy enough to securely hold post-surgical drains or other objects without additional assistance.

The garment is closed in the front using a secure closure sewn into or attached to the garment. In an example embodiment, a VELCRO® hook and loop closure, zipper, snaps, ties, eye hook and loop closure, or other secure closure is used.

In an example embodiment, the garment has an elastic band, cord, tie, or other material sewn to or attached to the top and/or bottom of the garment. In an example embodiment, the elastic bands secure the top and bottom of the garment to the patient. In another example embodiment, the garment is double lined, with the lining and the outer material sewn together with elastic, cord, ties or other material sewn or attached to the top and bottom of the garment. In an example embodiment, the garment is tied on the top and bottom without a front secure closure. In another example embodiment, the garment is tied on the top without a front secure closure.

Pouches or pockets are sewn onto or attached to the front, sides, and/or rear of the garment. In an example embodiment, one pouch is sewn onto the right front of the garment and one pouch is sewn into the left front of the garment. In an example embodiment, the pouches are made of the same lightweight or disposable fabric/material as the garment. In another example embodiment, the pouches are made of a SMS, Tyvek, poly-Lycra fabric blend, spandex, cotton, nylon, or other breathable material. In an example embodiment, the pouches are large enough to securely hold one or more post-surgical drains, catheters, colostomy bags, or other medical bags, collection containers, or tubing. In an example embodiment, the pouches cover and conceal the post-surgical drains. In an example embodiment, the pouches are made of a material that is lightweight and disposable, but sturdy enough to securely hold post-surgical drains or other objects without additional assistance.

In another example embodiment, the pouches are fully or partially removable. In an example embodiment, the pouches are attached to the garment by a VELCRO® hook and loop closure, zipper, snaps, ties, eye hook and loop closure, or other secure fastener on at least one side of the pouch. In another example embodiment, the pouches are sewn to the garment on at least one side of the pouch.

In an example embodiment, each pouch has an elastic band, cord, tie, or other material sewn or attached to the top of each pouch. In an example embodiment, the material secures the contents of the pouch.

In another example embodiment, a flap is sewn onto the pouch at the top. In an example embodiment, the flap closes or covers the pouch. In another example embodiment, the flap is sewn onto the side or bottom of the pouch. In an example embodiment, the flap is securely sewn on one side, allowing the user to open and close the flap as needed. In an example embodiment, the flap is made of the same lightweight or disposable fabric/material as the pouch and garment. In another example embodiment, the flap is secured on the remaining three sides using a secure closure sewn into or attached to the garment or pouch and into the flap. In another example embodiment, the flap is sewn or attached onto the pouch or garment on two or more sides and the flap is secured on the remaining sides using the secure closure. In an example embodiment, a VELCRO® hook and loop closure, zipper, snaps, ties, or other secure closure is used.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

FIG. 1 is an example plan view of a post-surgical drain management garment, in accordance with certain example embodiments.

FIG. 2 is an example side plan view of a post-surgical drain management garment, in accordance with certain example embodiments.

FIG. 3 is an example front/side perspective view of a post-surgical drain management garment worn around the waist of a patient, in accordance with certain example embodiments.

FIG. 4 is an example front/side perspective view of a post-surgical drain management garment worn around the knee of a patient, in accordance with certain example embodiments.

### DETAILED DESCRIPTION OF THE EXAMPLE EMBODIMENTS

The functionality of the various example embodiments will be explained in more detail in the following description, read in conjunction with the figures illustrating the example embodiments. Turning now to the drawings, in which like numerals indicate like (but not necessarily identical) elements throughout the figures, example embodiments are described in detail.

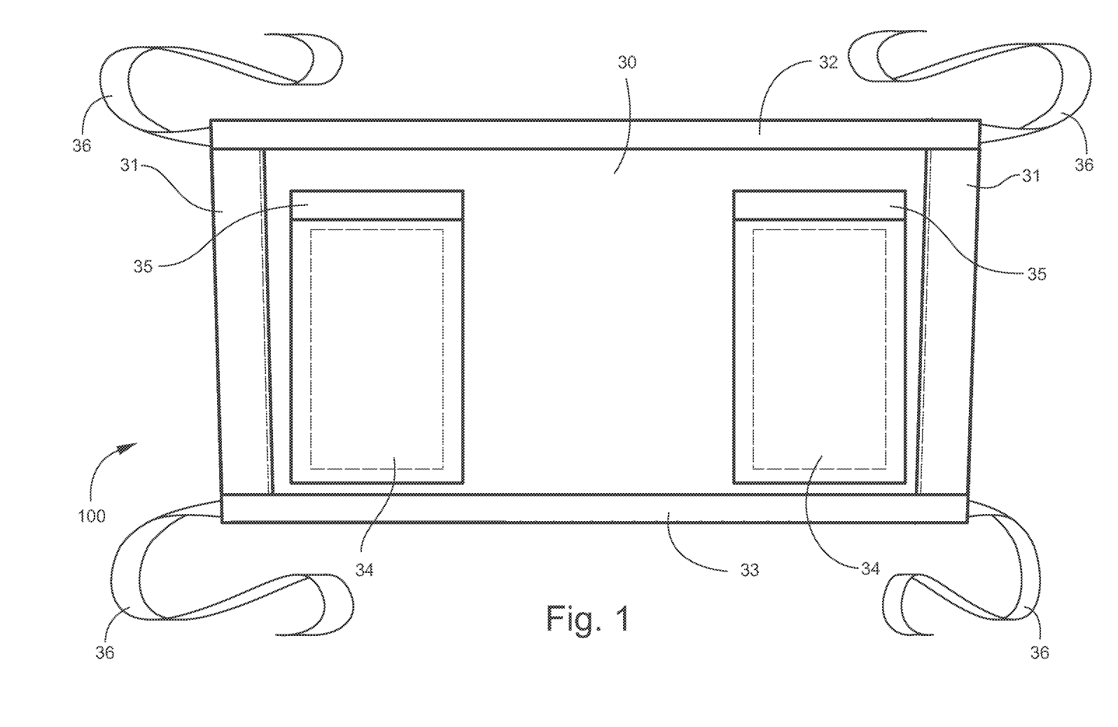

FIG. 1 is an example plan view of a post-surgical drain management garment 100, in accordance with certain example embodiments. As depicted in FIG. 1, the example post-surgical garment 100 comprises a wrap-around component made of a lightweight or disposable material 30, sewn-in front closure 31, a top closure assembly trim 32 at the top of the garment 100, a bottom closure assembly 33 at the bottom of the garment 100, one or more pouches/pockets 34, and an elastic trim 35 at the top of each of the pockets 34.

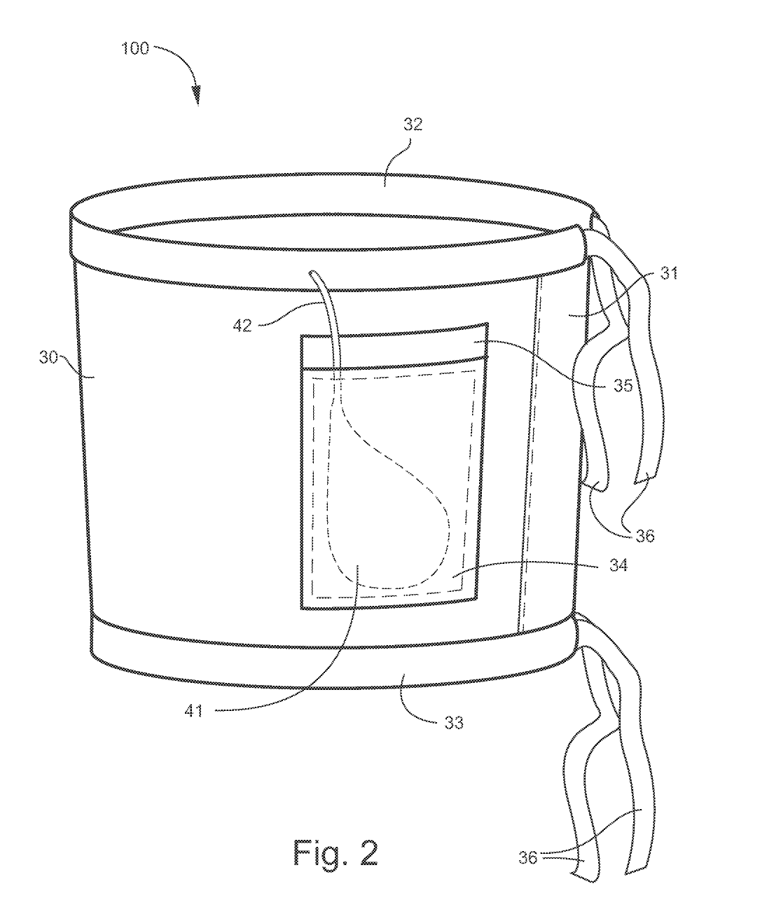

FIG. 2 is an example side plan view of the post-surgical drain management garment 100, in accordance with certain example embodiments. As depicted in FIG. 2, the example post-surgical drain management garment 100 comprises one or more pouches/pockets 34 with an elastic trim 35 at the top of each of the pockets 34. One or more post-surgical drains 41 and one or more corresponding sets of post-surgical drain tubing 42 are secured in the one or more pockets 34.

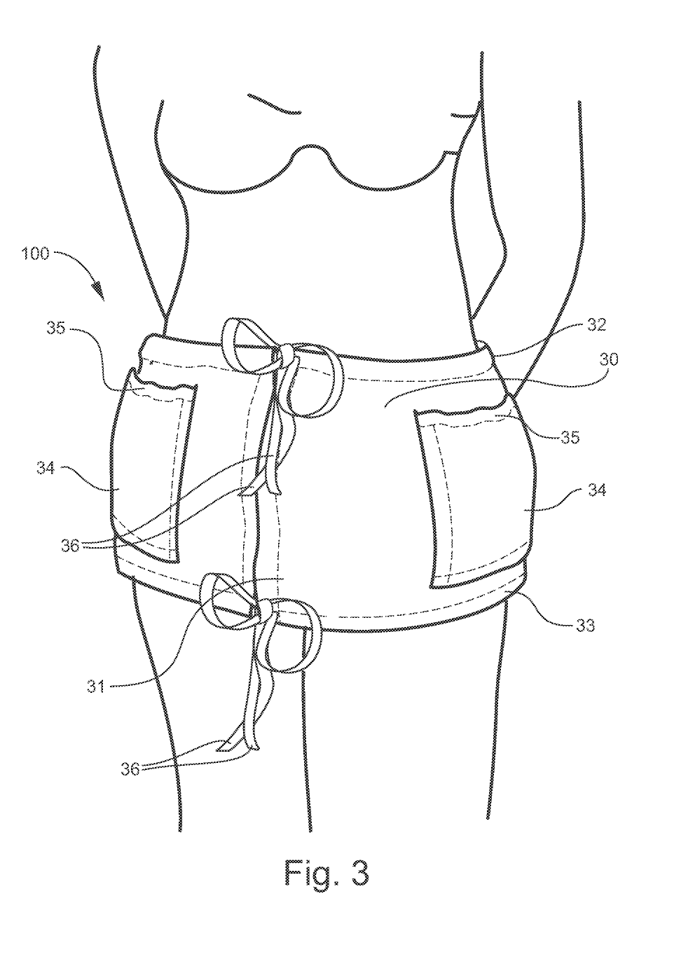

FIG. 3 is an example front/side perspective view of the post-surgical drain management garment 100 worn around the waist of a patient, in accordance with certain example embodiments. The example front/side perspective view of the post-surgical drain management garment 100 worn around the waist of a patient includes features described herein with reference to the components illustrated in FIGS. 1 and 2.

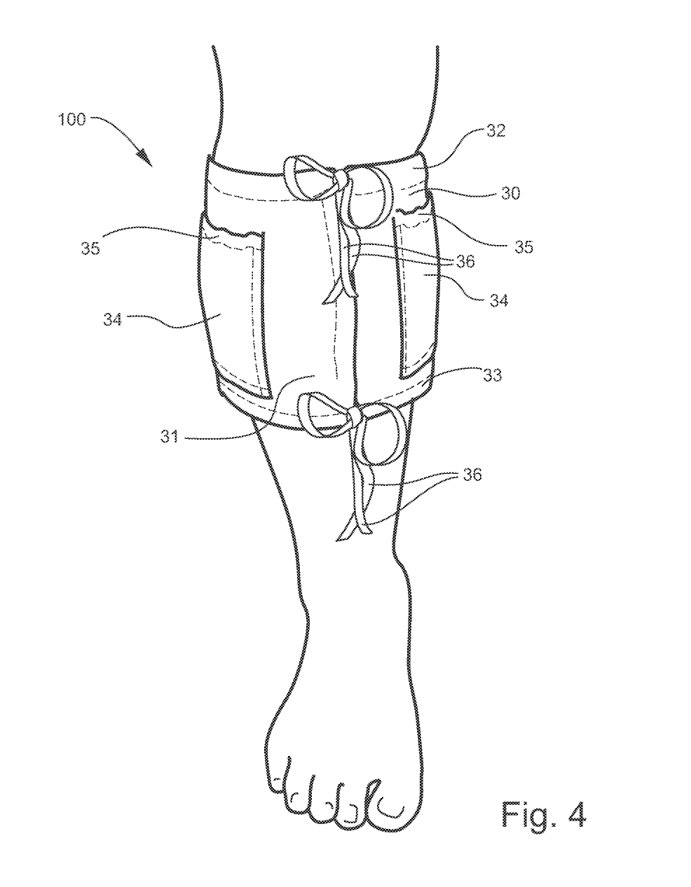

FIG. 4 is an example front/side perspective view of the post-surgical drain management garment 100 worn around the knee of a patient, in accordance with certain example embodiments. The example front/side perspective view of the post-surgical drain management garment 100 worn around the knee of a patient includes features described herein with reference to the components illustrated in FIGS. 1 and 2.

The components of the post-surgical garment 100 are described hereinafter with reference to the example embodiments.

In an example embodiment, a solid, wrap-around component 30 is made of a lightweight, disposable material. In another example embodiment, the wrap-around component 30 is made of a lightweight, breathable material. In yet another example embodiment, the wrap-around component 30 is made of a poly-Lycra fabric blend, spandex, cotton, nylon, or other breathable material. In an example embodiment, the wrap-around component 30 is loose fitting against the body and does not impede or restrict the wound area. In another example embodiment, the wrap-around component 30 matches the curvature of the body without covering, impeding, or restricting the wound area. In an example embodiment, the wrap-around component 30 is sturdy enough to support the weight of one or more post-surgical drains 41 without additional support or assistance when worn by a patient.

The wrap-around component 30 is connected with an adjustment mechanism 31, for example, sewn in or attached VELCRO® hook and loop closure, zipper, ties, buttons, or other closure. In an example embodiment, the adjustable closure mechanism 31 is situated in the front of the garment 100. In another example embodiment, the adjustable closure mechanism 31 is situated in the back or on the side of the garment 100 and can depend on how the patient wears the garment.

In an example embodiment, the wrap-around component 30 is lined at the top and bottom with closures assemblies 32, 33. In an example embodiment, the closures 32, 33 are sewn in or attached elastic, cord, ties, trim, or other material that can match curvature of the body. In an example embodiment, the closures assemblies 32, 33 lie loosely against the curvature of the body.

In another example embodiment, the closures assemblies 32, 33 comprise one or more ties 36. In this embodiment, the garment 100 comprises a sewn edge on the top and/or bottom of the wrap-around component 30 through which the ties 36 are fed through so that one end of the ties 36 lies on each side of the wrap-around component 30. Once the wrap-around component 30 is wrapped around the body of the patient, the ends of the ties 36 are knotted or tied together to secure the garment 100 to the body. In an example embodiment, the ties allow the patient to secure a wrap-around component 30 that is larger than the circumference of the patient's body. In this embodiment, the material of the wrap-around component 30 is bunched-up or pressed together on the ties 36. In another example embodiment, the ties 36 allow the patient to secure a wrap-around component 30 that is smaller than the circumference of the patient's body. In this embodiment, the ties 36 are long enough to wrap around the patient's body to secure the garment 100 to the body, but the material of the wrap-around component 30 is unable to wrap around the patient's body.

In an example embodiment, one or more pouches 34 are provided, between the bottom closure assembly 33 and the top closure assembly 32. The number of pouches 34 can vary. In an example embodiment, the pouches 34 are provided on each side of the garment 100. For example, two pouches 34 are situated independently on each side of the closure mechanism 31 on the wrap-around component 30 of the garment 100 in which to place the post-surgical drains 41. In an example embodiment, one or more post-surgical drains 41 fit in each pouch 34. In another example embodiment, each pouch 34 is large enough to securely hold one or more post-surgical drains 41, catheters, colostomy bags, or other medical bags, collection containers, and/or tubing 42. In an example embodiment, the pouches 34 are made of a lightweight, disposable material that is sturdy enough to securely hold the post-surgical drains 41 without human assistance or additional support. In another example embodiment, the pouches 34 are made of a lightweight, breathable material that is sturdy enough to securely hold the post-surgical drains 41 without human assistance or additional support. In yet another example embodiment, the pouches 34 are made of a poly-Lycra fabric blend, spandex, cotton, nylon, or other breathable material.

In an example embodiment, the pouches 34 cover and conceal the post-surgical drains 41. Each pouch 34 can be lined at the top with a closure assembly 35, for example, an elastic band, cord, or other material sewn to or attached to the top of the pouch 34 or to the garment above the pouch 34. In an example embodiment, the closure assembly 35 secures the contents of the pouch 34. In another example embodiment, the closure assembly 34 comprises ties or other type of trim/closure that aides in keeping the contents of the pouch 34 securely inside the pouch 34. In an example embodiment, the post-surgical drain 41 is slipped over the top or under the bottom of the wrap-around component 30 and inserted into the pouch 34.

In an example embodiment, each pouch 34 is sewn onto or attached to the wrap-around component 30 on three sides and the remaining side is open to allow the post-surgical drains 41 to be inserted between the material of the pouch 34 and the wrap-around component 30. In another example embodiment, the pouch 34 is securely sewn on one side and two sides are secured using a secure closure. In an example embodiment, a VELCRO® hook and loop closure, zipper, button, tie or other closure is used. The remaining side is open to allow the post-surgical drains 41 to be inserted between the material of the pouch 34 and the wrap-around component 30. In yet another example embodiment, the pouch 34 is securely sewn on two sides and one side is secured using a secure closure. In an example embodiment, a VELCRO® hook and loop closure, zipper, button, tie or other closure is used. The remaining side is open to allow the post-surgical drains 41 to be inserted between the material of the pouch 34 and the wrap-around component 30.

In an example embodiment, the wrap-around component 30 is wrapped around the user's torso, arm, leg, or other part of the body and secured with the closure 31 in the front, back, or side. The user places the post-surgical drain 41 over or under the wrap-around component 30 and secures it in the pouch 34. In an example embodiment, one post-surgical drain 41 is used per pouch 34. In another example embodiment, two or more drains 41 are used per pouch 34.

In another example embodiment, the wrap-around component 30 is wrapped around the user's torso, arm, leg, or other part of the body and secured with the ties 36. The user places the post-surgical drain 41 over or under the wrap-around component 30 and secures it in the pouch 34. In an example embodiment, one post-surgical drain 41 is used per pouch 34. In another example embodiment, two or more drains 41 are used per pouch 34.

### General

To facilitate an understanding of the principles and features of the various embodiments of the invention, various illustrative embodiments are explained above. Although exemplary embodiments of the invention are explained in detail, other embodiments are contemplated. Accordingly, it is not intended that the invention is limited in its scope to the details of construction and arrangement of components set forth in the description or illustrated in the drawings. The garment may be used by adults and children.

As used in the specification and the appended claims, the singular forms “a,” “an” and “the” include plural references unless the context clearly dictates otherwise. For example, reference to a component is intended also to include composition of a plurality of components.

Also, in describing the exemplary embodiments, terminology will be resorted to for the sake of clarity. Each term contemplates its broadest meaning as understood by those skilled in the art and includes all technical equivalents that operate in a similar manner to accomplish a similar purpose.

The mention of one or more method steps does not preclude the presence of additional method steps or intervening method steps between those steps expressly identified.

The materials described as making up the various elements of the invention are illustrative and not restrictive. Many suitable materials that would perform the same or a similar function as the materials described herein are encompassed within the scope of the invention. Such other materials not described herein can include, but are not limited to, for example, materials that are developed after the time of the development of the invention.

The example components, construction, and methods described in the embodiments presented previously are illustrative, and, in alternative embodiments, certain components can be placed in a different position, combined with another component, and/or omitted entirely, and/or certain additional components can be added, without departing from the scope and spirit of various embodiments as defined in the claims, the scope of which is to be accorded the broadest interpretation so as to encompass such alternatives.

Although specific embodiments have been described above in detail, the description is merely for purposes of illustration. It should be appreciated, therefore, that many aspects described above are not intended as required or essential elements unless explicitly stated otherwise. Modifications of, and equivalent components or acts corresponding to, the disclosed aspects of the example embodiments, in addition to those described above, can be made by a person of ordinary skill in the art, having the benefit of the present disclosure, without departing from the spirit and scope of embodiments defined in the following claims, the scope of which is to be accorded the broadest interpretation so as to encompass such modifications and equivalent structures.

What is claimed is:

1. A post-surgical materials management garment, comprising:
a body section of material that wraps around a user;
a top securing mechanism disposed on a top of the material that secures the garment around the user; and
one or more pouches on the external side of the garment in which one or more post-surgical materials is placed.

2. The post-surgical garment of claim 1, wherein the material comprises a lightweight, disposable material that supports a weight of the one or more post-surgical materials.

3. The post-surgical garment of claim 1, wherein the post-surgical garment is used while the user is in a hospital, recovery facility, or post-surgical facility.

4. The post-surgical garment of claim 1, further comprising a front closure mechanism that secures the garment around the user.

5. The post-surgical garment of claim 1, wherein the one or more pouches each comprise a lightweight, disposable material that supports the weight of the one or more post-surgical material.

6. The post-surgical garment of claim 1, wherein at least one the one or more pouches is securely attached to the body section of material on at least one side of the pouch and attached on at least one other side using a pouch closure mechanism.

7. The post-surgical garment of claim 1, wherein a pouch securing mechanism is disposed on a top edge of each of the one or more pouches that secures the one or more post-surgical materials in the pouch.

8. The post-surgical garment of claim 7, wherein the pouch securing mechanism comprises an elastic material coupled to the top edge of the pouch.

9. The post-surgical garment of claim 1, wherein the post-surgical materials comprise one or more of a post-surgical drain, a catheter, a colostomy bag, a collection bag, a collection containers, and a tubing.

10. The post-surgical garment of claim 1, wherein at least one of the one or more pouches can hold more than one post-surgical material.

11. The post-surgical garment of claim 1, wherein the material wraps around the user's torso, arm, or leg.

12. The post-surgical garment of claim 1, wherein the top securing mechanism comprises a tie attached along material on the top edge of the garment.

13. The post-surgical garment of claim 1, wherein the top securing mechanism comprises an elastic material sewn into the top edge of the material.

14. The post-surgical garment of claim 1, further comprising a bottom securing mechanism disposed on a bottom of the material that secures the garment around the user.

15. A method of securing post-surgical drains, comprising:
wrapping a material of a post-surgical drain management garment around a user;
securing the material around the user with a closure mechanism attached to the material of the post-surgical drain-management garment;
passing a post-surgical drain over the material of the post-surgical drain-management garment wrapped around a user; and
inserting the post-surgical drain into a pouch attached to the material.

16. The method of claim 15, wherein the material comprises a lightweight, disposable material that supports a weight of the one or more post-surgical materials.

17. The method of claim 15, wherein the post-surgical garment is used while the user is in a hospital, recovery facility, or post-surgical facility.

18. The method of claim 15, wherein the pouch comprises a lightweight, disposable material that supports a weight of the one or more post-surgical materials.

19. The method of claim 15, wherein the wrapping the material of the post-surgical drain management garment around the user comprises wrapping the material around the user's torso, arm, or leg.

20. The method of claim 15, wherein the pouch can hold more than one post-surgical drain.

21. The method of claim 15, wherein the closure mechanism comprises a top securing mechanism disposed on a top of the post-surgical drain management garment around the user.

22. The method of claim 15, wherein the closure mechanism comprises a front securing mechanism disposed of on the side edges of the post-surgical drain management garment around the user.
